(19) United States
(12) Patent Application Publication
(10) Pub. No.: US 2025/0269098 A1
(43) Pub. Date: Aug. 28, 2025

(54) Dialyzer for Blood Treatment and Corresponding System

(71) Applicant: Fresenius Medical Care Deutschland GmbH, Bad Homburg (DE)

(72) Inventors: Kee Jor, Bad Homburg (DE); Jiaming Dong, Bad Homburg (DE); Yicheng Peng, Bad Homburg (DE)

(21) Appl. No.: 18/858,100

(22) PCT Filed: Apr. 21, 2022
(86) PCT No.: PCT/CN2022/088100

§ 371 Date: Oct. 18, 2024

(51) Int. Cl. A61M 1/14; A61M 1/16

(52) U.S. Cl. CPC A61M 1/1565; A61M 1/154; A61M 1/1621; A61M 2205/3317; A61M 2205/3334

(57) ABSTRACT

Herein disclosed is a dialyzer, comprising: a housing defining a cavity comprising a first cavity region and a second cavity region which are communicated with each other by a pass-through passage; a plurality of hollow fiber membranes extending from the first cavity region to the second cavity region across the passage; a flow restricting structure located in an area of the passage so as to restrict flow of medical fluid, for example dialysate, between the first cavity region and the second cavity region; a first port and a second port each fluidly communicated with the first cavity region; and a third port and a fourth port each fluidly communicated with the second cavity region. Also disclosed is a corresponding system, comprising: the dialyzer described above, a blood line, and a medical fluid line, wherein the blood line and medical fluid line are fluidly connected with the dialyzer.

### CROSS-REFERENCE TO RELATED APPLICATIONS

The present application is the national stage entry of International Patent Application No. PCT/CN2022/088100, filed on Apr. 21, 2022, the disclosure of which is incorporated herein by reference.

### TECHNICAL FIELD

The disclosure relates to a dialyzer for blood treatment, and a corresponding system for blood treatment.

### BACKGROUND

A dialysis treatment is a procedure for removing toxic substances and metabolites normally removed by the kidneys, and for aiding in regulation of fluid and electrolyte balance.

The dialysis treatment may be carried out by various types of dialysis procedures, such as a hemodialysis (HD) and a peritoneal dialysis (PD). The hemodialysis is usually executed by using a hemodialysis machine.

Standard hemodialysis (HD) generally uses diffusion to remove waste products from a patient's blood. A diffusive gradient that occurs within the semi-permeable dialyzer between the blood and dialysate may cause diffusion. Further, hemofiltration (HF) is an alternative renal replacement therapy that relies on a convective transport of toxins from the patient's blood. This therapy is accomplished by directly or indirectly adding substitution or replacement fluid to the extracorporeal circuit during treatment. That substitution fluid and the fluid accumulated by the patient between dialysis sessions is ultrafiltered over the course of the HF treatment, thereby providing a convective transport mechanism that is beneficial in removing middle and large molecules.

Hemodiafiltration (HDF) is a treatment mode that combines convective and diffusive clearances and thus may represent a gold standard in the field of replacement of renal function by dialysis. HDF uses dialysate to flow through a dialyzer, for example a high flux dialyzer, similar to standard hemodialysis, providing diffusive clearance. In addition, the substitution solution, which also may be dialysate, is provided directly or indirectly to the extracorporeal circuit, providing convective clearance.

As a key part, the dialyzer may need to be further improved to simplify the structure and reduce the cost, especially for HDF. In some instances, there may be a need to provide an improved dialyzer which is more applicable to different blood treatment modes.

### SUMMARY

This disclosure relates to an improved dialyzer for blood treatment, and an improved system for blood treatment.

According to a first aspect, provided is a dialyzer for blood treatment, comprising: a housing defining a cavity comprising a first cavity region and a second cavity region which are communicated with each other by a pass-through passage; a plurality of hollow fiber membranes extending from the first cavity region to the second cavity region across the passage; a flow restricting structure located in an area of the passage so as to restrict flow of medical fluid, for example dialysate, between the first cavity region and the second cavity region; a first port and a second port each fluidly communicated with the first cavity region; and a third port and a fourth port each fluidly communicated with the second cavity region.

According to an optional embodiment of the present disclosure, the passage is configured as a narrowed region defined at least partially by the flow restricting structure; and/or the dialyzer is configured without any potting in the area of the passage.

According to an optional embodiment of the present disclosure, the flow restricting structure is configured to be disposed within the housing or to be integrally formed on the housing; and/or the flow restricting structure is designed while considering expansion property of the hollow fiber membranes in use; and/or the flow restricting structure is configured to relatively tightly hold the hollow fiber membranes. The term “relatively tightly hold the hollow fiber membranes” means, that the majority of the medical fluid flowing along the first and second cavity region is not passed through the flow restricting structure. In a further improved embodiment less than 20%, in some implementations less than 10% of the medical fluid is passed through the restricting structure.

According to an optional embodiment of the present disclosure, the flow restricting structure is configured to be formed during or after injection molding of the housing.

According to an optional embodiment of the present disclosure, the flow restricting structure comprises a protrusion formed during injection molding of the housing; or the flow restricting structure is formed at least partially by a shrinkable structure which can be shrunk after injection molding of the housing, in some implementations, after loading of the hollow fiber membranes.

According to an optional embodiment of the present disclosure, the protrusion is formed by a curved wall portion of the housing; or the shrinkable structure comprises a heat-shrinkable tube.

According to an optional embodiment of the present disclosure, the heat-shrinkable tube forms at least a portion of the housing or disposed within the housing.

According to an optional embodiment of the present disclosure, the first port or the second port is fluidly connected with the third port or the fourth port through a fluid flow control device to control flow of the medical fluid therebetween.

According to an optional embodiment of the present disclosure, the housing has a substantially cylindrical configuration and the first port, the second port, the third port and the fourth port are disposed on the housing sequentially in an axially spaced manner from each other in an axial direction, In some implementations the third and the fourth port are disposed in a row.

According to an optional embodiment of the present disclosure, the second port and the third port are fluidly connected by the fluid flow control device.

According to an optional embodiment of the present disclosure, the fluid flow control device comprises an on-off valve and/or a flow restriction.

According to an optional embodiment of the present disclosure, the on-off valve and the flow restriction are connected in parallel between the second port and the third port; and/or the on-off valve is a solenoid valve; and/or the flow restriction is a variable flow restriction; and/or the fluid flow control device further comprises a flow sensor for detecting a flowrate of the medical fluid between the second port and the third port.

According to an optional embodiment of the present disclosure, the fluid flow control device is integrated as a shunt interlock assembly.

According to a first aspect, provided is a system for blood treatment, comprising: the dialyzer described above, a blood line and a medical fluid line, wherein the blood line and medical fluid line are fluidly connected with the dialyzer.

According to an optional embodiment of the present disclosure, the system is configured to perform hemodialysis, hemofiltration, hemodiafiltration and any combination thereof.

According to some exemplary embodiment of the present disclosure, the dialyzer has a simplified structure and a low cost, and can achieve better control to adapt to various blood treatment modes.

### BRIEF DESCRIPTION OF THE DRAWINGS

The disclosure and advantages thereof will be further understood by reading the following detailed description of some exemplary embodiments with reference to the drawings, in which:

FIG. 1 schematically shows a sectional view of a dialyzer according to an exemplary embodiment of the present disclosure.

FIG. 2 schematically shows a sectional view of the dialyzer according to another exemplary embodiment of the present disclosure.

### DETAILED DESCRIPTION

Some exemplary embodiments of the present disclosure will be described hereinafter in more details with reference to the drawings to better understand the basic concept and advantages of the present disclosure.

According to an aspect of the disclosure, herein firstly proposed is a dialyzer for blood treatment, comprising: a housing defining a cavity comprising a first cavity region and a second cavity region which are communicated with each other by a pass-through passage; a plurality of hollow fiber membranes extending from the first cavity region to the second cavity region across the passage; a flow restricting structure located in an area of the passage so as to restrict flow of medical fluid, for example dialysate, between the first cavity region and the second cavity region; a first port and a second port fluidly communicated with the first cavity region; and a third port and a fourth port fluidly communicated with the second cavity region.

Here, both the first cavity region and the second cavity region are formed within a common housing.

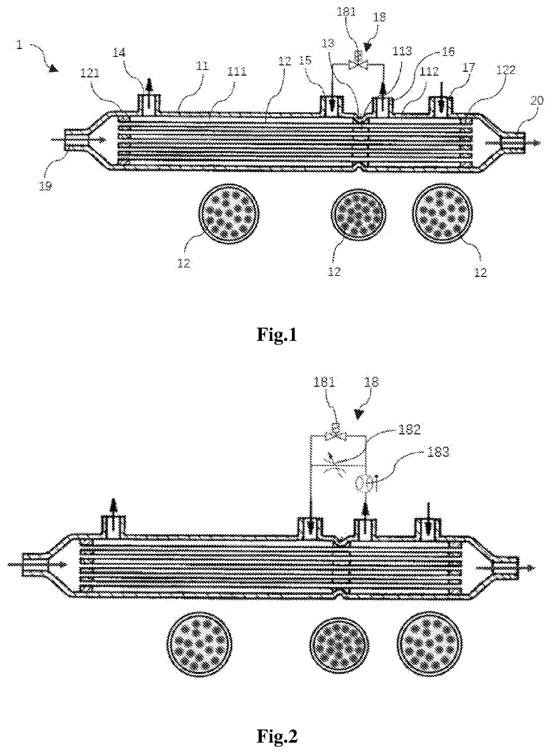

FIG. 1 schematically shows a sectional view of the dialyzer 1 according to an exemplary embodiment of the present disclosure.

As shown in FIG. 1, the dialyzer 1 may comprise: a substantially cylindrical housing 11 defining a cavity comprising a first cavity region 111 and a second cavity region 112 which may be communicated with each other by a pass-through passage 113, wherein the first cavity region 111, the second cavity region 112 and the pass-through passage 113 may be arranged coaxially; a plurality of hollow fiber membranes 12 extending from the first cavity region 111 to the second cavity region 112 across the passage 113; a flow restricting structure 13 formed in an area of the passage 113 so as to restrict flow of the medical fluid from the second cavity region 112 to the first cavity region 111; a first port 14, as a dialysate (effluent) outlet port, also called as an ultrafiltrate port in some cases, and a second port 15, as a dialysate inlet port, fluidly communicated with the first cavity region 111; and a third port 16, as a further dialysate outlet port, and a fourth port 17, as a further dialysate inlet port, fluidly communicated with the second cavity region 112. In this case, the dialyzer 1 actually comprises two sub-dialyzers using the common housing 11.

The dialyzer 1 may have a blood inlet port 19 and a blood outlet port 20. During the blood treatment, the blood to be treated flows into the blood inlet port 19 via an arterial blood line (not shown) from a patient, flow through lumens of the hollow fiber membranes 12, and then the treated blood flows out of the blood outlet port 20 via a venous blood line (not shown) back to the patient, while the dialysate flows in the first cavity region 111 and the second cavity region 112. Due to the fact that the basic working principle of the dialyzer 1 itself during the blood treatment is known, the description will mainly focus on some parts associated with the present disclosure to achieve a corresponding simplification.

In FIG. 1, as shown in corresponding arrows, the dialysate flows within the housing 11 in a countercurrent manner, which can achieve better blood treatment, including with more intense diffusion across the hollow fiber membranes 12. However, the skilled person in the art may understand that this is just an example.

As shown in FIG. 1, the hollow fiber membranes 12 are potted only at their opposite ends 121 and 122. That means that each of the hollow fiber membranes 12 is continuous across the passage 113 without any potting area in the area of the passage 113.

According to an exemplary embodiment of the present disclosure, as shown in FIG. 1, the passage 113 may be configured as a narrowed region defined at least partially by the flow restricting structure 13. In this narrowed region, the hollow fiber membranes 12 are denser due to radial compression on them.

FIG. 1 also shows cross-sectional views of the dialyzer 1 at three corresponding different areas in its lower portion. As can be seen from FIG. 1, the hollow fiber membranes 12 are closer to each other in the area of the passage 113, which means that the dialysate is more difficult to, and even cannot, flow through gaps between any adjacent two of the hollow fiber membranes 12.

According to an exemplary embodiment of the present disclosure, as shown in FIG. 1, the flow restricting structure 13 may be configured to be integrally formed on the housing 11. As can be seen from FIG. 1, the flow restricting structure 13 is formed as a portion of the housing 11. In this case, the flow restricting structure 13 may be formed during injection molding of the housing 11 (which is often made of plastic). As shown in FIG. 1, the flow restricting structure 13 may comprise a protrusion formed during injection molding of the housing 11. The protrusion can be formed by a curved wall portion of the housing 11. Such a configuration may be formed easily during injection molding of the housing 11.

The skilled person in the art may understand that the flow restricting structure 13 also may be configured to be disposed within the housing 11. In some implementations, the flow restricting structure 13 may be configured as a separate part and then be positioned relative to, for example be attached to the housing 11 in any suitable manner. For example, the hollow fiber membranes 12 may be loaded within the flow restricting structure 13 and then they together be loaded into the housing 11.

According to an exemplary embodiment of the present disclosure, at least one, and in some implementations all, of the first port 14, the second port 15, the third port 16 and the fourth port 17 may be molded integrally with the housing 11, which is very advantageous.

However, if the flow restricting structure 13 is pre-molded as shown in FIG. 1, it may be difficult to load the hollow fiber membranes 12 through the passage 113 due to a limited size of the passage 113. Thus, it may be advantageous that the flow restricting structure 13 is formed finally at least after the hollow fiber membranes 12 are loaded into the cavity of the housing 11.

Thus, according to an exemplary embodiment of the present disclosure, the flow restricting structure 13 may be formed at least partially by a shrinkable structure which can be shrunk after injection molding of the housing 11, including in some instances, after loading of the hollow fiber membranes 12.

In some implementations, the shrinkable structure may be a heat-shrinkable structure, such as a heat-shrinkable tube. Shrinking of the shrinkable structure can be preformed easily for the heat-shrinkable structure. Of course, the present disclosure is not limited hereto. For example, the shrinkable structure can be shrunk by lights having a specific wave length.

According to an exemplary embodiment of the present disclosure, the heat-shrinkable tube may form at least a portion, for example an axial segment of the housing 11 in the area of the passage 113. That is, at least this segment may be formed by a heat-shrinkable material.

The skilled person in the art may understand that the heat-shrinkable tube may be firstly disposed within the housing 11 and then be shrunk, in some instances after loading of the hollow fiber membranes 12.

Generally, the hollow fiber membranes 12 will expand when exposed to water, which means that on the one hand the flow restricting structure 13 should be designed while considering expansion property of the hollow fiber membranes 12 in use to avoid excessive compression of the hollow fiber membranes 12 and thus damage to the hollow fiber membranes 12 or adversely affecting flowing of the blood through the lumens of the hollow fiber membranes 12, but on the other hand, we can take advantage of this to help to restrict flow of the medical fluid from the second cavity region 112 to the first cavity region 111, additionally considering that a priming process should be performed before starting the actual blood treatment.

Thus, it may be sufficient to configure the flow restricting structure 13 to relatively tightly hold the hollow fiber membranes 12 as shown in FIG. 1.

According to an exemplary embodiment of the present disclosure, the first port 14 or the second port 15 may be connected with the third port 16 or the fourth port 17 through a fluid flow control device 18 to control flow of the medical fluid therebetween. FIG. 1 just shows an example in which the second port 15 and the third port 16 are connected by the fluid flow control device 18.

The dialyzer 1 has the first cavity region 111 and the second cavity region 112, between which the flow restricting structure 13 is located, such that pressure profiles of the dialysate within the first cavity region 111 and the second cavity region 112 can be controlled or adjusted in a more flexible manner to achieve a desired treatment goal, which may vary with different treatment conditions, particularly for different patients. Thus, the present disclosure does not intend to expel any other possibilities, for example connecting of the third port 16 with the first port 14, although the specific connection manner as shown in FIG. 1 may be advantageous for most of blood treatment modes.

As shown in FIG. 1, the first port 14, the second port 15, the third port 16 and the fourth port 17 may be disposed on the housing 11 sequentially in an axially spaced manner from each other in an axial direction, in some implementations in a row. In some implementations, at least one, or all, of the first port 14, the second port 15, the third port 16 and the fourth port 17 may extend laterally, as shown in FIG. 1.

As can be seen from FIG. 1, the second port 15 and the third port 16 may be disposed at opposite side of the flow restricting structure 13 respectively and are adjacent to each other.

According to an exemplary embodiment of the present disclosure, as shown in FIG. 1, the fluid flow control device 18 may comprise an on-off valve 181, which can switch on or off flowing of the dialysate from the third port 16 toward the second port 15.

According to an exemplary embodiment of the present disclosure, the on-off valve 181 may be a solenoid valve, which can be controlled easily as desired. In this case, the solenoid valve can be controlled flexibly in on/off cycles.

However, such a control manner may cause undesirable momentary stress across the hollow fiber membranes 12. In this case, it may be advantageous to provide a flow restriction 182 connected in parallel with the on-off valve 181, which is exemplarily shown in FIG. 2, which is the same as FIG. 1 except for the different fluid flow control device 18. The flow restriction 182 can partially divert some dialysate and thus be effective to lower momentary peak pressure acting on the hollow fiber membranes 12. Use of the flow restriction 182 also can optimize the HDF treatment process.

The skilled person in the art may understand that the flow restriction 182 also may be used instead of the on-off valve 181. Only use of the flow restriction 182 also can achieve the HDF treatment mode.

According to an exemplary embodiment of the present disclosure, the flow restriction 182 may be a variable flow restriction. In some implementations, the variable flow restriction can be controlled automatically.

As shown in FIG. 2, according to an exemplary embodiment of the present disclosure, the fluid flow control device 18 may further comprise a flow sensor 183 for detecting a flowrate of the dialysate from the third port 16 to the second port 15. In some implementations, the flow sensor 183 may be disposed upstream of the on-off valve 181 and the flow restriction 182.

According to an exemplary embodiment of the present disclosure, the fluid flow control device 18 may be integrated as a shunt interlock assembly, which will facilitate assembling of the fluid flow control device 18 onto the second port 15 and the third port 16.

A controller (not shown) may be provided to control the fluid flow control device 18 to adapt to various blood treatments.

According to another aspect of the present disclosure, further proposed is a system for blood treatment, comprising the dialyzer 1 described above, a blood line (not shown) and a medical fluid line (not shown), wherein the blood line and medical fluid line are fluidly connected with the dialyzer 1.

According to an exemplary embodiment of the present disclosure, the system may be configured to perform hemodialysis, hemofiltration, hemodiafiltration and any combination thereof.

While certain embodiments have been described, these embodiments have been presented by way of example only, and are not intended to limit the scope of the disclosure. The attached claims and their equivalents are intended to cover all the modifications, substitutions and changes as would fall within the scope and spirit of the disclosure.

1-15. (canceled)

16. A dialyzer for blood treatment, comprising:
a housing defining a cavity comprising a first cavity region and a second cavity region which are communicated with each other by a pass-through passage;
a plurality of hollow fiber membranes extending from the first cavity region to the second cavity region across the passage;
a flow restricting structure located in an area of the passage so as to restrict flow of medical fluid, for example dialysate, between the first cavity region and the second cavity region;
a first port and a second port each fluidly communicated with the first cavity region; and
a third port and a fourth port each fluidly communicated with the second cavity region.

17. The dialyzer according to claim 16, wherein
the flow restricting structure is configured to be formed during or after injection molding of the housing.

18. The dialyzer according to claim 17, wherein
the flow restricting structure comprises a protrusion formed during injection molding of the housing, or
the flow restricting structure is formed at least partially by a shrinkable structure which can be shrunk after injection molding of the housing, particularly after loading of the hollow fiber membranes.

19. The dialyzer according to claim 18, wherein
the protrusion is formed by a curved wall portion of the housing; or
the shrinkable structure comprises a heat-shrinkable tube.

20. The dialyzer according to claim 19, wherein
the heat-shrinkable tube forms at least a portion of the housing or disposed within the housing.

21. The dialyzer according to claim 16, wherein
the passage is configured as a narrowed region defined at least partially by the flow restricting structure; and/or
the dialyzer is configured without any potting in the area of the passage.

22. The dialyzer according to claim 21, wherein
the flow restricting structure is configured to be disposed within the housing or to be integrally formed on the housing; and/or
the flow restricting structure is designed while considering expansion property of the hollow fiber membranes in use; and/or
the flow restricting structure is configured to relatively tightly hold the hollow fiber membranes.

23. The dialyzer according to claim 21, wherein
the flow restricting structure is configured to be formed during or after injection molding of the housing.

24. The dialyzer according to claim 21, wherein
the first port or the second port is fluidly connected with the third port or the fourth port through a fluid flow control device to control flow of the medical fluid therebetween.

25. The dialyzer according to claim 16, wherein
the flow restricting structure is configured to be disposed within the housing or to be integrally formed on the housing; and/or
the flow restricting structure is designed while considering expansion property of the hollow fiber membranes in use; and/or
the flow restricting structure is configured to relatively tightly hold the hollow fiber membranes.

26. The dialyzer according to claim 25, wherein
the first port or the second port is fluidly connected with the third port or the fourth port through a fluid flow control device to control flow of the medical fluid therebetween.

27. The dialyzer according to claim 25, wherein
the flow restricting structure is configured to be formed during or after injection molding of the housing.

28. The dialyzer according to claim 16, wherein
the first port or the second port is fluidly connected with the third port or the fourth port through a fluid flow control device to control flow of the medical fluid therebetween.

29. The dialyzer according to claim 28, wherein
the housing has a substantially cylindrical configuration and the first port, the second port, the third port and the fourth port are disposed on the housing sequentially in an axially spaced manner from each other in an axial direction, preferably in a row.

30. The dialyzer according to claim 29, wherein
the second port and the third port are fluidly connected by the fluid flow control device.

31. The dialyzer according to claim 28, wherein
the fluid flow control device comprises an on-off valve and/or a flow restriction.

32. The dialyzer according to claim 31, wherein
the on-off valve and the flow restriction are connected in parallel between the second port and the third port; and/or
the on-off valve is a solenoid valve; and/or
the flow restriction is a variable flow restriction; and/or
the fluid flow control device further comprises a flow sensor for detecting a flowrate of the medical fluid between the second port and the third port.

33. The dialyzer according to claim 28, wherein
the fluid flow control device is integrated as a shunt interlock assembly.

34. A system for blood treatment, comprising:
the dialyzer according to claim 16;
a blood line; and
a medical fluid line;
wherein the blood line and medical fluid line are fluidly connected with the dialyzer.

35. The system according to claim 34, wherein
the system is configured to perform hemodialysis, hemofiltration, hemodiafiltration and any combination thereof.
